(19) United States
(12) Patent Application Publication
(10) Pub. No.: US 2010/0248361 A1
(43) Pub. Date: Sep. 30, 2010

(54) PLATELET PRODUCTION METHODS

(75) Inventors: Larry C. Lasky, Columbus, OH (US); Brent Sullenbarger, Dayton, OH (US); Nicholas A. Kotov, Ypsilanti, MI (US)

Correspondence Address: CALFEE HALTER & GRISWOLD, LLP, 800 SUPERIOR AVENUE, SUITE 1400, CLEVELAND, OH, 44114, US

(73) Assignees: THE OHIO STATE UNIVERSITY RESEARCH FOUNDATION, Columbus, OH (US); THE REGENTS OF THE UNIVERSITY OF MICHIGAN, Ann Arbor, MI (US)

(21) Appl. No.: 12/730,504

(22) Filed: Mar. 24, 2010

Related U.S. Application Data
(60) Provisional application No. 61/162,857, filed on Mar. 24, 2009.

(51) Int. Cl. C12N 5/078

(52) U.S. Cl. 435/355; 435/372

(57) ABSTRACT

The present invention provides platelet production methods. The method comprises the steps of providing a cellular material and culturing the cellular material, wherein platelets are produced. The culturing may be performed on 2D or 3D cell support structure or in suspension culture. In some embodiments, all or a part of the 2D or 3D culturing may be performed in a bioreactor. In some embodiments, the method may further comprise a step of isolating a subset of cells from the starting cellular material, wherein the isolated subset of cells is then cultured, wherein platelets are produced. In yet other embodiments, the method comprises the steps of providing a cellular material, isolating a subset of cells, seeding the subset of cells into a 3D scaffold, culturing the subset of cells in a 3D scaffold, seeding the cultured subset of cells into a bioreactor, culturing the subset of cells in a bioreactor, and harvesting the cells from the bioreactor, wherein platelets are produced.

### CROSS-REFERENCE TO RELATED APPLICATIONS

This application claims the benefit of U.S. Provisional Application No. 61/162,857, filed on Mar. 24, 2009, hereby incorporated by reference in its entirety.

### STATEMENT REGARDING FEDERALLY SPONSORED RESEARCH

The work described in this application was supported, at least in part, by NIH 5R01 EB007350-02 and NIH R21 HL072088. The United States government may have certain rights in this invention.

### BACKGROUND

A method to produce transfusable platelets in vitro would obviate many of the problems encountered with current methods to procure this life-saving blood component. Published studies of in vitro megakaryocyte or platelet production have described the use of several starting cell populations including human cord blood, embryonic stem cells, cell lines, peripheral blood progenitor cells, and marrow [1-13]. Many of these described production of megakaryocytes but not platelets. Some were performed in suspension culture or on plastic surfaces; some used feeder layers. A wide variety of added growth factors, often including thrombopoietin (TPO), stem cell factor (SCF) and Flt-3 ligand (FL), or conditioned medium have been used. Several included IL-6 or IL-11 in the last stage of the culture, and found that this increased the number of putative platelets produced [1]. Studies of platelet production per se were performed mainly to shed light on the platelet production process itself, without quantification of numbers produced. Gandhi et al, for example, described production of platelets both from a megakaryocytic cell line and from marrow in tissue culture flasks [11]. The platelets produced aggregated when exposed to appropriate agonists.

Early hematopoietic cells are adherent to stromal cells and extracellular matrix in vivo; this adherence influences their ability to self-renew. The earliest hematopoietic cells are found in niches along the bone in marrow spaces, relying on cell-cell interactions and the local milieu to determine their immediate fate [14]. Some aspects of the 3D niche microenvironment can be supplied by providing a feeder cell layer. These cells may supply points of contact for adherent cells, or molecules that interact with receptors on the target cell's surface and may produce soluble chemokines or cytokines necessary for growth and survival of target cells. Several cell lines have been developed for growth of hematopoietic cells, notably the HS-5 line [15]. Primary marrow stromal cells (MSC) also have been used for this purpose, partially recapitulating the role of these cells in vivo [16, 17]. One group has reported growing marrow CD34 positive cells on MSCs, with formation of megakaryocyte colonies in serum-free medium without added cytokines [18].

While Masunaga et at found that they could generate platelets using stromal support for CD34+ cell expansion into megakaryocytes, others have found the opposite. For instance, Zweegman et at found that stromal proteoglycans bound the megakaryocytopoiesis-inhibiting cytokines IL-8 and MIP-1 [24]. Supporting the use of stromal cells, Cheng et at found that they could produce platelets in culture from CD34 positive cells grown on human MSC, using serum-free medium without added growth factors [18]. Tablin and colleagues found that guinea pig proplatelets form and release platelets well from megakaryocytes on rat tail collagen gel, but are disrupted growing on Matrigel [25].

### BRIEF SUMMARY

The present invention provides platelet production methods. The method comprises the steps of providing a cellular material and culturing the cellular material, wherein platelets are produced. The culturing may be performed on 2D or 3D cell support structure or in suspension culture. In some embodiments, all or a part of the 2D or 3D culturing may be performed in a bioreactor. In some embodiments, the method may further comprise a step of isolating a subset of cells from the starting cellular material, wherein the isolated subset of cells is then cultured, wherein platelets are produced. In yet other embodiments, the method comprises the steps of providing a cellular material, isolating a subset of cells, seeding the subset of cells into a 3D scaffold, culturing the subset of cells in a 3D scaffold, seeding the cultured subset of cells into a bioreactor, culturing the subset of cells in a bioreactor, and harvesting the cells from the bioreactor, wherein platelets are produced. In some embodiments, all or some of the steps of isolating a subset of cells, seeding the subset of cells into a 3D scaffold, culturing the subset of cells in a 3D scaffold, seeding the cultured subset of cells into a bioreactor, culturing the subset of cells in a bioreactor, and harvesting the cells from the bioreactor may be repeated as necessary. In other embodiments, all of the steps of isolating a subset of cells, seeding the subset of cells into a 3D scaffold, culturing the subset of cells in a 3D scaffold, seeding the cultured subset of cells into a bioreactor, culturing the subset of cells in a bioreactor, and harvesting the cells from the bioreactor may be repeated as necessary.

Platelets prepared by a process comprising: providing a cellular material and culturing the cellular material are also contemplated. The culturing may be performed on 2D or 3D cell support structure or in suspension culture. In some embodiments, all or part of the 2D or 3D culturing may be performed in a bioreactor. In some embodiments, the process may further comprise a step of isolating a subset of cells from the starting cellular material, wherein the isolated subset of cells is then cultured. In yet other embodiments, the process comprises the steps of providing a cellular material, isolating a subset of cells, seeding the subset of cells into a 3D scaffold, culturing the subset of cells in a 3D scaffold, seeding the cultured subset of cells into a bioreactor, culturing the subset of cells in a bioreactor, and harvesting the cells from the bioreactor. In some embodiments, some of the steps of isolating a subset of cells, seeding the subset of cells into a 3D scaffold, culturing the subset of cells in a 3D scaffold, seeding the cultured subset of cells into a bioreactor, culturing the subset of cells in a bioreactor, and harvesting the cells from the bioreactor may be repeated as necessary. In other embodiments, all of the steps of isolating a subset of cells, seeding the subset of cells into a 3D scaffold, culturing the subset of cells in a 3D scaffold, seeding the cultured subset of cells into a bioreactor, culturing the subset of cells in a bioreactor, and harvesting the cells from the bioreactor may be repeated as necessary.

The present invention contemplates the use of a starting cellular material, wherein platelets are produced. The cellular material may be progenitor cellular material. The progenitor cellular material may be pluripotent stem cells. The pluripotent stem cells may be selected from the group consisting of derived stem cells, harvested stem cells, and embryonic stems cells. In some embodiments, the harvested stem cells may be selected from the group consisting of induced adult stem cells, genetically modified stem cells, bone marrow stem cells, peripheral blood stem cells, fetal liver stem cells, and umbilical cord blood stem cells. In some embodiments, the derived stem cells, harvested stem cells, and embryonic stems cells may be selected from CD34+ or CD133+ cells. In some embodiments, the CD34+ or CD133+ cells may be selected from hematopoietic stem cells (HSCs), multipotent progenitor cells (MPPs), and lineage-restricted progenitor cells (LRPs). In some embodiments, the HSCs are CD150⁺CD48⁻ CD244⁻. In some embodiments, the MPPs are CD150⁻CD48⁺CD244⁺. In some embodiments, the LRPs are CD150⁻CD48⁺CD244⁺. In some embodiments, the HSCs are selected from mouse HSC, human HSC, LT-HSC, and ST-HSC. Mouse HSC may be CD34^{lo/-}, SCA-1⁺, Thy1.1^{+/lo}, CD38⁺, C-kit⁺, lin⁻. Human HSC may be CD34⁺, CD59⁺, Thy1/CD90⁺, CD38^{lo/-}, C-kit/CD117⁺, lin⁻. LT-HSC may be CD34⁻, SCA-1⁺, Thy1.1^{+/lo}, C-kit⁺, lin⁻, CD135⁻, Slamf1/CD150⁺. ST-HSC may be SCA-1⁺, Thy1.1^{+/lo}, C-kit⁺, lin⁻, CD135⁻, Slamf1/CD150⁺, Mac-1 (CD11b)^{lo}. In some embodiments, the MPPs are selected from early MPP and late MPP. Early MPP may be CD34⁺, SCA-1⁺, Thy1.1⁻, C-kit⁺, lin⁻, CD135⁺, Slamf1/CD150⁻, Mac-1 (CD11b)^{lo}, CD4^{lo}. Late MPP may be CD34⁺, SCA-1⁺, Thy1.1⁻, C-kit⁺, lin⁻, CD135^{high}, Slamf1/CD150⁻, Mac-1 (CD11b)^{lo}, CD4^{lo}.

Culturing conditions for the present invention may be ex vivo culturing conditions. In some embodiments, the ex vivo culturing conditions may utilize a humidified atmosphere, maintained thermal conditions, and a cell culture media. Thermal conditions may be selected from normothermic (37° C.), hyperthermic (>37° C.), and hypothermic (<37° C.). Atmospheric conditions may be selected from: ambient humidified air, 5% CO₂; 5% oxygen humidified, 5% CO₂; and discontinuous 5% CO₂ ambient humidified air 5% CO₂. In some embodiments, the cell culture media may be selected to expand, maintain, differentiate, and produce platelets. In some embodiments, the media may be IMDM based culture media with cytokines and chemical messengers to expand, maintain, differentiate, and produce platelets. In some embodiments, the media is selected from expansion media, maintenance media, differentiation media, and platelet production media. Expansion media may comprise at least one of Iscove's modified Dulbecco's medium (IMDM), FCS, SCF, FL, TPO, and IL-6. Fusion proteins such as TAT-HOXB4 or NUP98-HOX made from self-renewal transcription factors such as HOXB4 may be included in the expansion media. [46], [48-50] In some embodiments, the expansion media may be serum free media. In some embodiments, the serum free media may comprise at least one of Iscove's modified Dulbecco's medium (IMDM), FCS, SCF, FL, TPO, and IL-6. In some embodiments, the expansion media may comprise IMDM, which may comprise at least one of 10% fetal calf serum, 50 ng/mL SCF, 50 ng/mL FL, 10 ng/mL TPO, and 10 ng/mL IL-6. In some embodiments, the serum free media may comprise at least one of 10% fetal calf serum, 50 ng/mL SCF, 50 ng/mL FL, 10 ng/mL TPO, and 10 ng/mL IL-6. The maintenance media may comprise at least one of IMDM, FCS, HS, HC, TPO, and FL. In some embodiments, the maintenance media may be serum free media. In some embodiments, the serum free media may comprise at least one of IMDM, FCS, HS, HC, TPO, and FL. In some embodiments, the maintenance medium may comprise IMDM, which may comprise at least one of 10% FCS, 10% horse serum (HS), 0.25 uM hydrocortisone (HC), 10 ng/mL TPO, and 25 ng/mL FL. In some embodiments, the serum free media may comprise at least one of 10% FCS, 10% horse serum (HS), 0.25 uM hydrocortisone (HC), 10 ng/mL TPO, and 25 ng/mL FL. The differentiation media may comprise at least one of IMDM, FBS, SRC, TPO, SCF, IL-6, and IL-9. In some embodiments, the differentiation medium may comprise IMDM, which may comprise at least one of 10% FBS, 30 ng/mL TPO, 1 ng/mL SCF, 7.5 ng/mL IL-6, 13.5 ng/mL IL-9, and 2.5 uM SU6656 (a Src kinase inhibitor). In some embodiments, the differentiation media may be serum free media. In some embodiments, the serum free media may comprise at least one of IMDM, FBS, SRC, TPO, SCF, IL-6, IL-9, and SU6656. The platelet production media may comprise at least one of IMDM, FBS, BSA, TPO, IL-3, IL-6, and IL-11. In some embodiments, the platelet production media may be serum free media. In some embodiments, the serum free media may comprise at least one of IMDM, FBS, BSA, TPO, IL-3, IL-6, and IL-11. In some embodiments, the platelet production medium may comprise IMDM, 10% FBS, 1% bovine serum albumin (BSA), 25 ng/mL TPO, 25 ng/mL IL-3, 50 ng/mL IL-6, and 10 ng/mL IL-11. In some embodiments, the serum free media may comprise at least one of IMDM, 10% FBS, 1% bovine serum albumin (BSA), 25 ng/mL TPO, 25 ng/mL IL-3, 50 ng/mL IL-6, and 10 ng/mL IL-11.

The method for producing platelets contemplates use of a cell support structure for cell culturing. In some embodiments, the cell support structure may be a 3D cell support structure. In some embodiments, the 3D cell support structure may be polymer based, ceramic/thin film, human or animal-derived, or fabric. The polymer based 3D cell support structure may be micro/macro porous. In some embodiments, it may be colloidal crystal. In other embodiments, it may be polyacrylamide hydrogel. The ceramic/thin film 3D cell support structure may be selected from medical ceramic, plastic, and bioglass. The plastic 3D cell support structure may be selected from monostructured thermomolded monofilm, polymer thin films, and tissue culture plastics. The fabric 3D cell support structure may be selected from woven and non-woven. In some embodiments, the woven fabric 3D cell support structure may be BARD. In some embodiments, the non-woven fabric 3D cell support structure may be PET.

The instant invention also contemplates the use of a bioreactor in the method for platelet production. The bioreactor may be selected from stirred tank suspension, fixed/fluidized bed, airlift, perfusion chamber, hollow fiber, packed bed, rotating wall vessel, gas permeable storage bag, stirred vessel, BioFlo, Celligen, and WaveBag. The bioreactor may be modular. Medium and nutrient low through the bioreactor may occur over the surface of the cell-scaffold construct, through the construct, or may comprise some combination of these methods of flow.

Additional features and advantages will be set forth in part in the description that follows, and in part will be obvious from the description, or may be learned by practice of the invention. The objects and advantages of the invention will be realized and attained by means of the elements and combinations throughout the disclosure.

It is to be understood that both the foregoing general description and the following detailed description are exemplary and explanatory only and are not restrictive of the invention, as claimed.

### BRIEF DESCRIPTION OF THE SEVERAL VIEWS OF THE DRAWING

The accompanying drawings, which are incorporated in and constitute a part of this specification, illustrate some embodiments of the invention, and together with the description, serve to explain principles of the invention.

FIG. 1 shows: (A) exploded-view cartoon of bioreactor module; (B) comparison of daily putative platelet production using the Src kinase inhibitor differentiation medium, results of three experiments (the differentiation medium was added on day 7); (C) flow cytometric analysis of shed cell output over time from the 3D bioreactor in Experiment 2 (differentiation medium was begun at day 7 (left column)) (in the control, expansion medium used through day 7 was continued (right column)) (forward scatter is plotted on the horizontal axes, and CD41 fluorescence is plotted on the vertical axes); (D) the number of putative platelets produced per day using the modular bioreactor system as in (B), but for this experiment we used 6 million CD34 positively-selected cord cells that had been expanded in brief liquid culture, putting 2 million cells in each of three bioreactor modules (Table 1); (E) Representative Wright's-stained microscopic (top) and TEM (bottom) images of platelets from the prototype modular bioreactor system (right) and fresh human cord blood platelets (left); and (F) we analyzed the platelets for CD62 and CD63 expression before and after thrombin exposure, and compared this to neonatal platelets harvested from cord blood within 24 hours of delivery, using flow cytometry.

FIG. 2 shows: (A) phase (first column), fluorescent (second column) and combined (third column) microscopic images of cells growing in hydrogel scaffold, cells are stained with FITC-labeled anti-CD41, the top row is a low power view that shows the shape of the interconnecting cavities in the scaffold and the large extent of megakaryocyte and platelet production in the scaffold, the lower row is a high power view of an isolated megakaryocyte on the wall of a cavity; and (B) daily putative platelet production in hydrogel scaffolds in wells: effect of protein coating, results are shown for Src kinase differentiation medium (left graph), and for IL-6,-11 differentiation medium after 7 days of Src differentiation medium (right graph).

FIG. 3 shows experimental entity-attribute relationship data model.

FIG. 4 shows increased putative platelet production using flow across cell-scaffold construct during differentiation phase. Horizontal axis: days; Vertical axis: Putative platelets produced per day. Bolus refers to 3 mL fresh medium feeding and cell collection performed every 24 hours (n=1).

### DETAILED DESCRIPTION

The present invention will now be described by reference to some more detailed embodiments, with occasional reference to the accompanying drawings. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention, the singular forms “a,” “an,” and “the” are intended to include the plural forms as well, unless the context clearly indicates otherwise. All publications, patent applications, patents, and other references mentioned herein are incorporated by reference in their entirety.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

Cord blood is used clinically as a source of hematopoietic progenitors. It is readily available and largely merely discarded after delivery. On the other hand, there are only a limited number of cells available in each collection. Disclosed herein is use of cord blood as a source of cells for production of platelets in vitro. It has been shown that growth in a 3D matrix of nonwoven polyester fabric enhances expansion of committed progenitors, compared to growth on a 2D surface [19]. In addition to using fibrous polyester 3D scaffolds described herein, use of purpose-built hydrogel scaffolds is disclosed. Colloidal crystals can be self-assembled by sedimentation/evaporation of corresponding dispersions and then annealed to form solids [20-23]. Inverted colloidal crystals (ICC) have unique advantages as cell culture substrates, including unprecedented level of control over the 3D geometry of cellular matrixes; high porosity (74% void volume); exceptionally high interconnectivity—each cavity has a total of 12 neighboring ones with 12 interconnecting channels; spheroid shape of the cavities hosting cells, stimulating intercellular interactions; and simplicity of preparation. ICC scaffolds can be made without any highly specialized equipment. Demonstrated herein is that utilization of this geometry constructed with biocompatible hydrogel provides a useful milieu for marrow cell growth and platelet production.

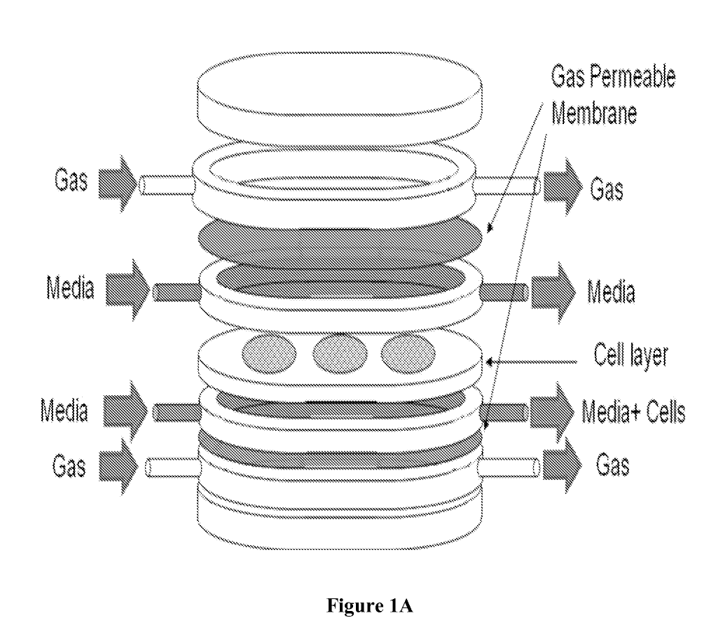

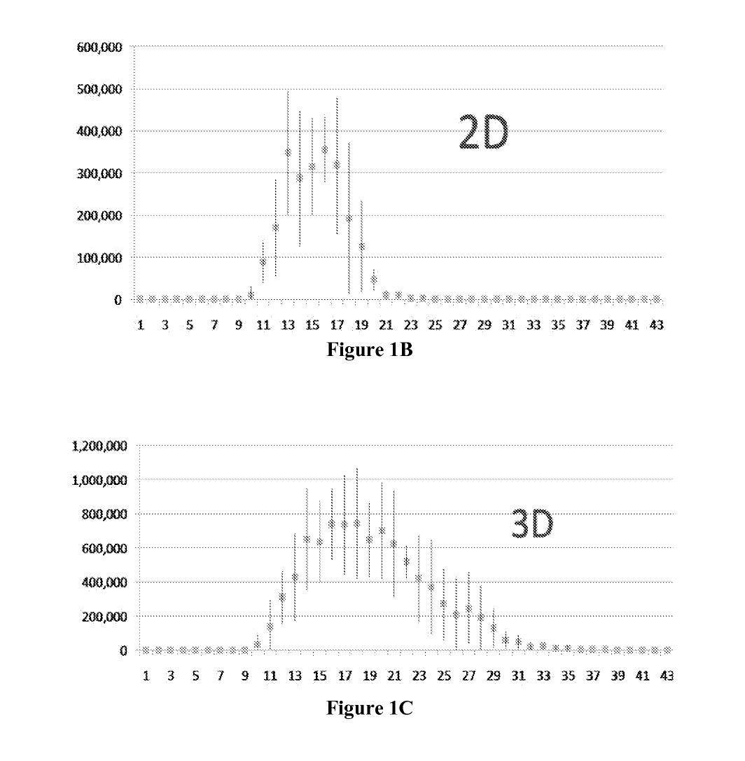

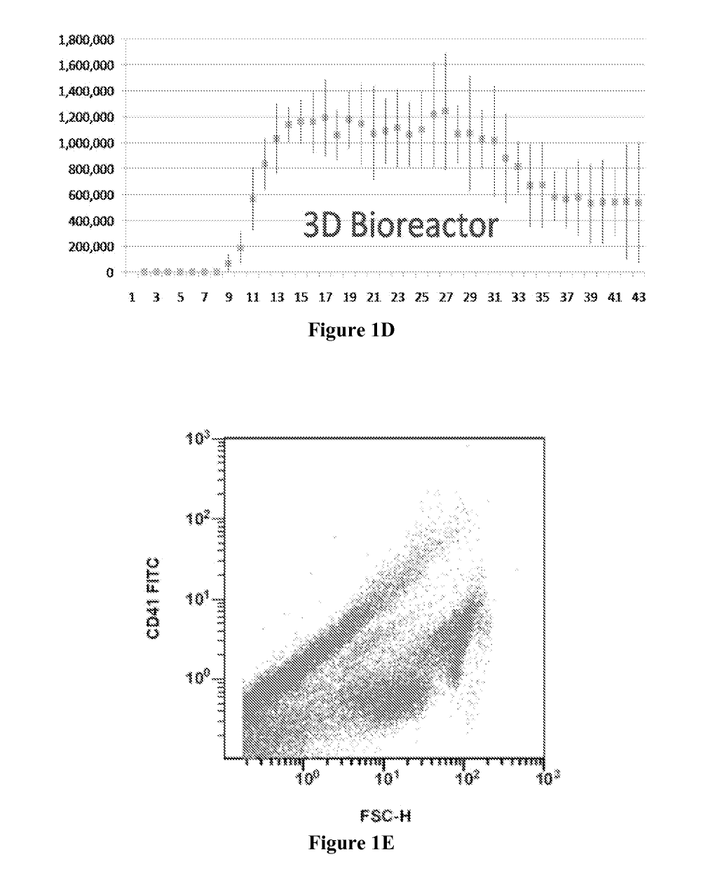

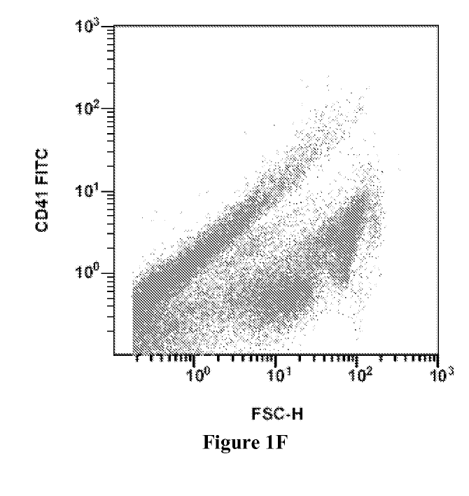

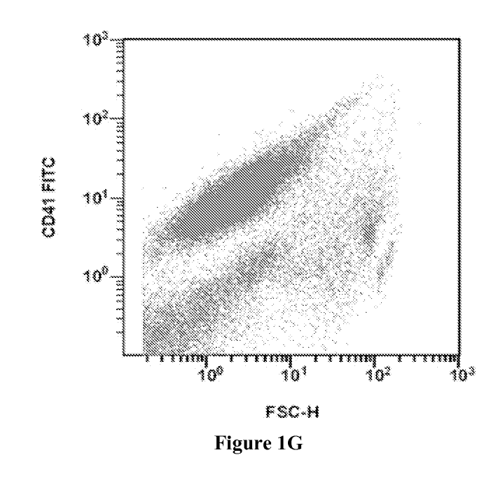

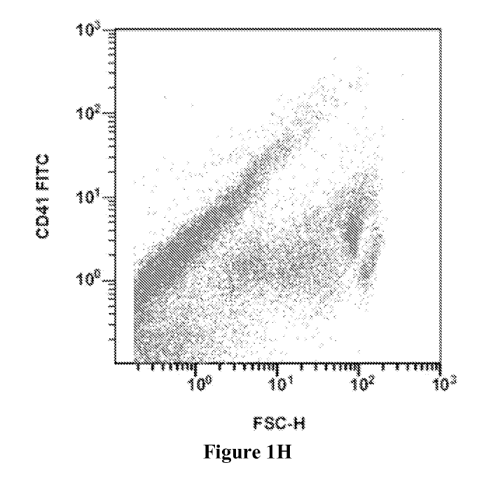

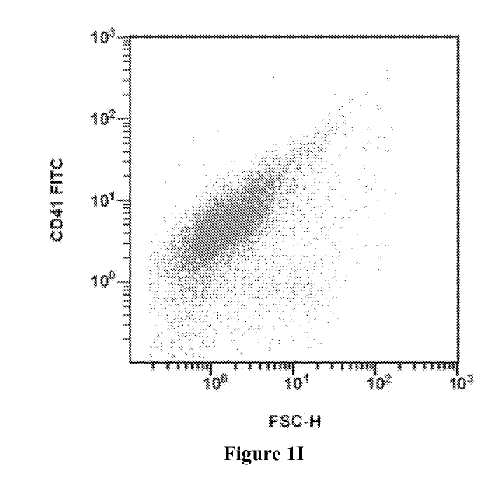

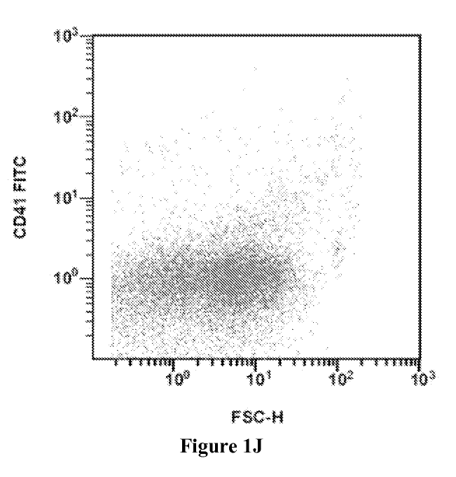

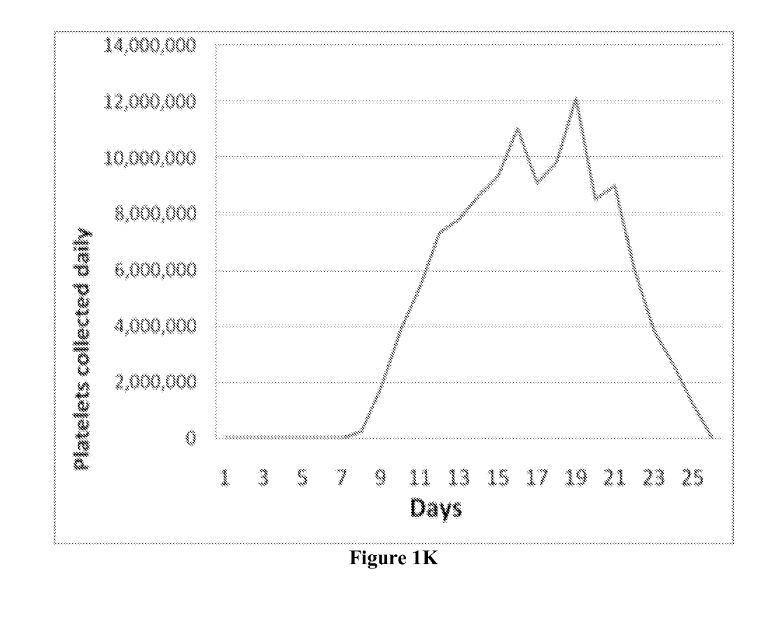

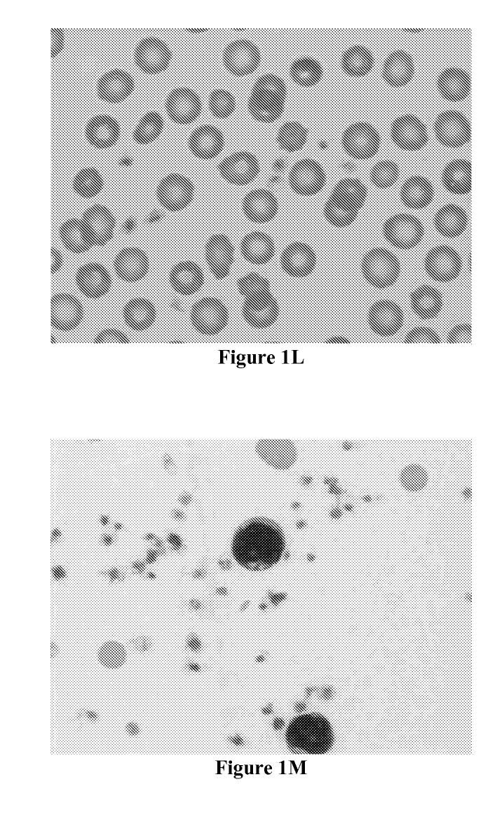

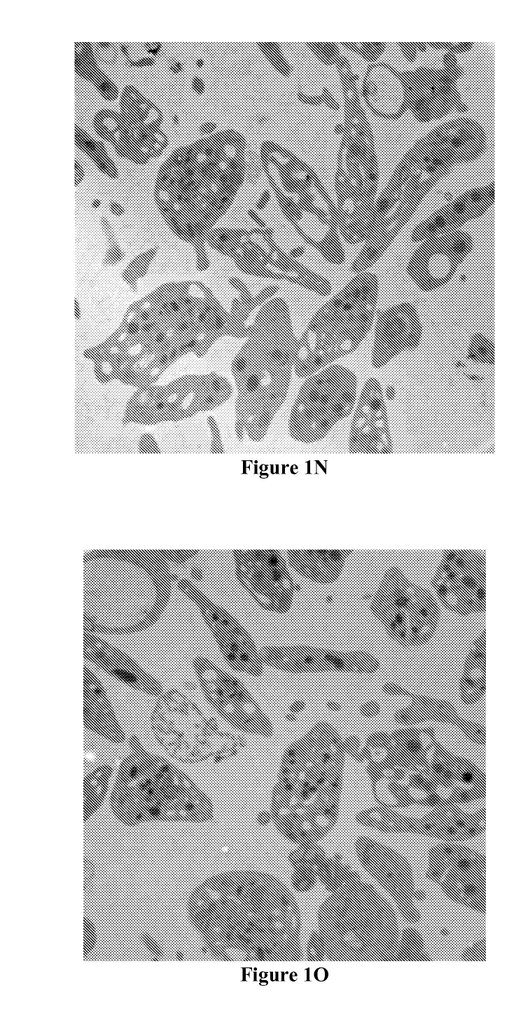

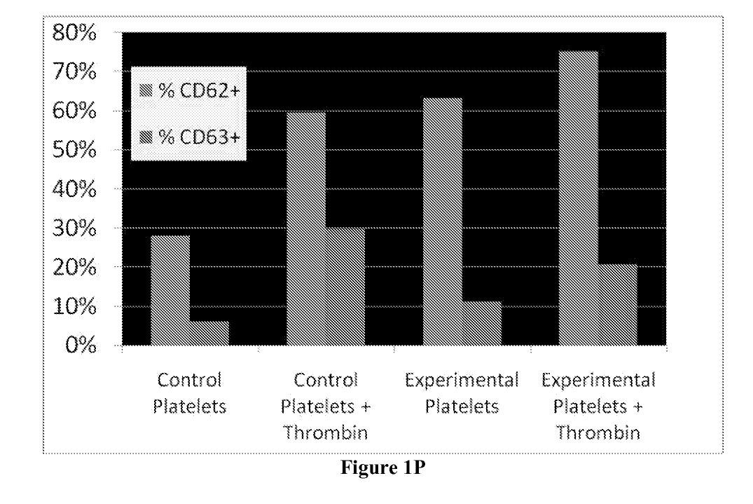

A number of bioreactor types have been used in hematopoietic stem cell expansion and research, including stirred tank suspension, fluidized bed, fixed bed, airlift, perfusion chamber, and hollow fiber [26]. The most frequently described, growth in (2D) static flasks or suspension, limit long term cell-cell or cell-matrix interactions. To see if a more physiologic milieu would further promote platelet production, we utilized woven polyester surgical fabric scaffold in a 3D single-pass perfusion bioreactor system. The bioreactor module is designed to allow maturing cells to settle continuously into the spent medium while immobilized parent cells are out of the direct medium flow path (FIG. 1A). The system is modular, and we have successfully used multiple bioreactor modules in parallel to facilitate cell production. This may avoid problems like those described by Matsunaga et al, in which they needed a very low concentration of early progenitors to produce large numbers of platelets in their feeder layer system [1]. Our system provides a large effective surface area, both as a result of the use of a 3D fabric scaffold and of the modular nature of the system.

### Materials & Methods

### CD34 Positively-Selected Cord Blood Cell Isolation

Umbilical cord blood units were obtained from normal full-term deliveries after institutional review board approval and informed consent. Light density cells were isolated from citrated cord blood using discontinuous density centrifugation using Ficoll-Paque Plus (GE Healthcare BioSciences, Uppsala, Sweden). CD34-positive selection was conducted using a MACS Direct CD34 Progenitor Cell Isolation Kit (Miltenyi Biotec, Anaheim, Calif.). In our laboratory this historically produces greater than 90% CD34 positive cells, as confirmed by flow cytometry. Only samples with greater than 95% viability as determined by trypan blue dye exclusion were used in further studies.

### 3D Scaffolds

Two types of scaffolds were used. The first was 1.9 cm diameter×1 mm depth disks of sterile surgical grade BARD polyester velour woven fabric (C. R. Bard, Inc., Humacao, Puerto Rico). This was used in both 12 well plates and the perfusion bioreactor system.

The second scaffold type was constructed from hydrogel. For manufacture of these scaffolds, colloidal crystal (CC) was used as a template for the 3D poly(acrylamide(Am)) hydrogel cell culture scaffold. The fabrication protocol of the CC resembles the process developed by Cuddihy and Kotov, and its transformation to inverted colloidal crystal (ICC) hydrogel scaffold resembles the process developed by Lee et al [21, 27]. The CC was constructed by sedimentation of soda lime glass spheres while providing agitation via sonication. To further ensure a high degree of orderliness, the sedimentation rate was retarded by focusing the spheres into a narrow channel prior to entering the mold and by the use of ethylene glycol (Sigma, St Louis, Mo.) as the sedimentation medium. After the thickness of the CC grew to a desired height, the CC was dried at 160° C. and annealed at 700° C. for 4 hours. The heat treatment caused partial melting at the surfaces, which resulted in annealing of spheres with their adjacent neighbors.

Upon the fabrication of colloidal crystal, the poly(Am) hydrogel precursor, composed of a 30 wt % acrylamide (Sigma) precursor containing 5 wt % of N,N-methylenebisacrylamide (NMBA) cross-linker, was infiltrated into the CC via centrifugation. Low viscosity of the precursor solution ensured complete infiltration in between the beads. Polymerization was initiated by adding aqueous potassium persulfate (KPS) solution (1 w/v %) at a ratio of 1:10 by volume in an oxygen-free environment. After polymerization, excess hydrogel pieces were removed and the hydrogel encapsulated CC was then immersed in a hydrofluoric acid (HF) and subsequent hydrochloric acid (HCl) bath to extract the internal glass spheres, resulting in a disc-shaped 3D ICC poly(Am) hydrogel scaffold. The ICC poly(Am) hydrogel scaffold was detoxified of the etchants by excessive rinsing with pH 10 buffer, 0.1 M calcium chloride solution and water followed by freeze drying.

The surfaces of ICC hydrogel scaffolds were modified through layer-by-layer deposition of positively charged 0.5 wt % poly(diallyldimethylammonium chloride) (PDDA, MW=200,000, Sigma) solution and negatively charged 0.5 wt % clay platelet (average 1 nm thick and 70-150 nm in diameter, Southern Clay Products) dispersion with deionized water rinse in between the steps. Duration of each deposition and rinse was 15 minutes and a cycle of PDDA adsorption/rinse/clay adsorption/rinse process was repeated 5 times. Following the deposition of PDDA, selected scaffolds were coated separately with using 1 mg/mL fibronectin in phosphate buffered saline (PBS) or 10 ug/mL TPO in PBS for 1 hour at room temperature, then washed in PBS to remove unbound protein.

The hydrogel scaffolds, 9 to 11 mm diameter disks approximately 1 mm thick, were used in well plates. Cavities in the hydrogel were 355 to 420 um in diameter, and they interconnected with adjacent cavities by openings that averaged 10 to 15% of the cavity diameter. They were placed on sterile polyester 1 mm mesh (Textile Development Associates, Franklin Square, N.Y.) over a conical funnel to facilitate seeding of cells into the hydrogel scaffolds by gravity filtration of cell-containing medium through the scaffold. After seeding, hydrogel scaffolds were placed in 24-well tissue culture plates and maintained in 1 mL medium at 37° C. humidified air 5% CO₂.

### 3D perfusion bioreactor system

The perfusion bioreactor modules are self-contained cell support systems that facilitate medium and gas exchange over and under a cell support scaffold (FIG. 1A). The device is constructed from polycarbonate. The gas-permeable membrane is made from smooth finish fluorinated ethylene propylene (McMaster Carr, Aurora, Ohio). Three disks of woven polyester fabric, 1.9 cm diameter and 1 mm thick, used as cell scaffolds, were fixed in the center. Medium flows over and under the disks, but not through them, minimizing shear forces. Non-adherent cells produced during incubation fell into the lower medium space, from which they were be collected in the discard medium pushed out of the bioreactor module when fresh medium is added. For gas exchange, the medium is separated by a sterile smooth-finish fluorinated ethylene propylene 0.025 mm thick gas-permeable membrane (McMaster Carr Aurora, Ohio) barrier from a continuous flow of humidified 5% CO₂ in air through the bioreactor module. Each bioreactor module has separate seeding and sampling ports allowing each module to be manipulated or removed independently of the other modules despite connections through the tubing network. In the experiments described, each module supported three BARD fabric cell scaffold disks. Bioreactor modules were run in parallel, with parallel bioreactor modules connected to a single fresh medium source.

To seed the cells into the modules, cell-containing medium was injected via a Luer lock connection. The tubing leading to and from the bioreactor module was clamped to create a cross flow of medium across the cell chamber containing the pre-placed fabric scaffold. The cells were then injected into the bioreactor module from the syringe such that the medium flow was through the matrix from top to bottom, resulting in trapping of cells within the scaffold. Cells were then permitted to adhere to the scaffold for 24-72 hours before the tubing clamps were removed and regular cross flow of medium resumed.

Medium exchange and cell harvests were accomplished simultaneously in the single pass bioreactor module. Daily, medium (3 mL) containing non-adherent cells were withdrawn from the bioreactor as fresh medium entered the module from the medium supply. For platelet function studies, a syringe containing 35 mL of glucose-based platelet storage medium, as previously described by Holme et al, was attached to the harvest port of the bioreactor module [28]. Three mL of platelet differentiation medium containing shed cells was withdrawn into the syringe.

### Medium and Growth Factors, Experimental Design

CD34-enriched cells were initially expanded in liquid culture at 37 C humidified air mixed with 5% CO₂ for 48-72 hours in Iscove's modified Dulbecco's medium (IMDM; Gibco, Invitrogen, Carlsbad, Calif.) expansion medium containing 10% fetal bovine serum (FCS; JRH Bio Sciences Lenexa, Kans.), 50 ng/ml SCF, 50 ng/ml FL, 10 ng/ml TPO, and 10 ng/ml IL-6. All growth factors were from R and D Systems, Minneapolis, Minn.

After the liquid culture expanded cells were seeded separately into each type of support scaffold. The cells were maintained for 7 days with daily medium changes after 48 hours in hematopoietic progenitor maintenance medium containing IMDM, 10% FCS, 10% horse serum (JRH Bio Sciences), 0.25 uM hydrocortisone, 10 ng/mL TPO, and 25 ng/mL FL. All additional medium additives and growth factors were from R and D Systems, Minneapolis. After 7 days, the medium was changed to differentiation medium to promote megakaryocyte and platelet production (Src differentiation medium) that consisted of IMDM, 10% FBS, 30 ng/ml TPO, 1 ng/ml SCF, 7.5 ng/ml IL-6, 13.5 ng/ml IL-9, and 2.5 uM SU6656 (Sigma), a Src kinase inhibitor.

This regime was used to compare incubation in tissue culture wells, in polyester fibrous scaffolds in wells, and in polyester fibrous scaffolds in the bioreactor system. Each experiment was performed using a unit of cord blood; approximately two million CD34 positively-selected cells were used for each of the three conditions. One aliquot was cultured at the bottom of 6 tissue culture wells in a 12 well plate, one was cultured in six 1.9 cm diameter×1 mm woven polyester scaffold maintained in 6 tissue culture wells in a 12 well plate, and the last was cultured in the 3D single pass perfusion bioreactor, using two modules, containing a total of 6 woven polyester scaffolds, connected in parallel to gas and medium supplies, and maintained at 37° C.

To determine the possible effect of increasing IL-6 and adding IL-11, one experiment was performed using IMDM containing 10% FBS, 1% bovine serum albumen, 25 ng/mL TPO, 25 ng/mL IL-3, 50 ng/mL IL-6, and 10 ng/mL IL-11 as differentiation medium (IL-6,-11 medium).

One experiment to compare effects of different protein coatings was performed in hydrogel scaffolds in tissue culture wells using Src differentiation medium. Then, to determine the possible effect of more IL-6 and added IL-11 in the protein-coated hydrogel scaffolds, another experiment using platelet differentiation medium containing IL-11 and a higher dose of IL-6 was performed. Src differentiation medium was used from day 7 to 14, when the differentiation medium was switched to IL-6,-11 medium on day 14.

### Imaging

Light microscopy was performed on cells harvested from culture conditions on an Axioskop 2 Zeiss microscope (Carl Zeiss, Thornwood, N.Y.) utilizing the Axiocam imaging system. Wright's-stained Cytospin (Shandon, Pittsburgh, Pa.) preparations were examined for morphology. For fluorescent studies of hydrogel scaffold sections, wedges were cut from whole scaffolds on day 32 following cell seeding. The hydrogel scaffold sections were incubated with FITC-labeled anti-CD41 or control IgG FITC antibodies, washed in PBS and placed on glass slides under Coverwell imaging chamber gaskets (Molecular Probes, Eugene Oreg.).

For transmission electron microscopy (TEM), an enriched platelet suspension was fixed in a 10:1 solution of phosphate buffered saline and 2.5% glutaraldehyde, pH 7.4. Following fixation, the platelet suspension was transferred to 2% liquefied agarose at 45° C. The agarose block containing the visible platelet pellet was then processed for TEM following the methods detailed by McLean et al with the exception that for fixation, an Epon-ethanol mixture was used instead of Spurr [29]. Studies were performed using a FEI Technai G2 Spirit Transmission Electron Microscope (Eindhoven, Netherlands).

### Flow Cytometry and Aggregation Studies

Cells for flow cytometric analysis were washed in Ca++/Mg++free Dulbecco's Phosphate Buffered Saline to remove culture medium and labeled with FITC/PE conjugated antibodies (Immunotech, Marseille, France) for surface antigens, including those for CD34, CD41, CD62, and CD63. Excess antibody was removed by washing, and the fluorescent cell analysis was performed on a BD FACS Calibur System flow cytometer (BD Biosciences, San Jose, Calif.). For comparison neonatal platelets were isolated from citrated cord blood by 120×g centrifugation for 15 minutes at room temperature. Neonatal platelets were then washed and labeled as described for experimentally-produced platelets.

To determine the functional properties of platelets harvested from 2D, 3D, and 3D perfusion bioreactor growth conditions, as well as hydrogel scaffolds, harvested platelets were collected, washed, and resuspended in 35 mL glucose storage buffer, incubated with or without 1.0 U/mL thrombin for 10 min at 37° C., and observed for aggregation using phase microscopy [28].

To detect platelet activation antigens CD62 and CD63, platelets were isolated and enriched as previously described. The platelet-rich portion was then resuspended in Ca++/Mg++ free Dulbecco's PBS and incubated with or without 1.0 U/mL thrombin for 10 min at 37° C. Platelet activation surface antigens CD62 and CD63 were measured by flow cytometry before and after exposure to thrombin.

### Results

### Comparison of Platelet Production in 2D, 3D, and Bioreactor

CD34 positively-selected cord cells were expanded for 48 to 72 hours in liquid culture. This increased the number of cells from about 2 million to about 15 million, and increased the number of CD34+ cells 4 to 5 fold. Following this, CD34 positively-selected cord cells from the same donor were divided in equal numbers among three conditions. Cells were further incubated in wells (2D), introduced into fabric scaffolds (3D), or infused into perfusion bioreactor modules containing identical scaffolds (3D bioreactor; Table 1). Daily, old medium was removed and fresh medium was added. Cells in the old medium from each condition were harvested by centrifugation, counted, and further characterized. We connected two identical bioreactor modules in parallel for these experiments. At 7 days, growth factors were changed to IL-6, IL-9, SCF, TPO, and SU6656, a Src kinase inhibitor. We used a modification of the method described by Gandhi et at for 2D culture [11]. We replaced IL-3 with IL-9 based on the work of Cortin et al, who carefully compared a large number of cytokine combinations for their megakaryocyte and platelet production potential, and that of Fujiki et at [30, 31]. In three identical experiments, in tissue culture wells (2D), 2.3, 1.8, and 1.7×10⁶ morphologic platelets were produced from day 12 to 25; in 3D scaffolds in wells (with a total of 6 disks per experiment), 10, 6.9, and 9.3×10⁶ from day 12 to 37; and in 3D scaffolds in the modular perfusion bioreactor, 31, 31, and 36×10⁶ from day 8 to day 40 (FIG. 1B). When normalized to number of platelets produced per CD34 positively-selected expanded cord blood cell, the 3D bioreactor produced statistically significantly higher yield per starting cell compared to either 2D or 3D production in wells.

### Platelet Production Scale-Up

When we increased the dose of IL-6 and added IL-11 we found a considerable increase in length of platelet production and numbers produced daily (Table 1 and FIG. 1D). For this experiment we used 6 million CD34 positively-selected cord cells that had been expanded in brief liquid culture, putting 2 million cells in each of three bioreactor modules (Table 1). Even considering the increased starting numbers over the previous experiments, the number of platelets produced per day was increased dramatically over the previously-used cytokine combination. Approximately twice the number of putative platelets per expanded CD34 positively-selected cell that we observed with Src differentiation medium was produced.

### Platelet Evaluation

Compound microscopy of Wright-stained smears and Cytospins showed a heterogeneous mixture of normal and atypically-shaped and -sized platelets (FIG. 1E). TEMs showed the presence of alpha and dense granules, mitochondria, open cannicular elements, and circumferential microtubules similar to that seen with concurrent fresh cord blood platelets, although there were somewhat more microparticles, ghosts, and abnormally-shaped and -sized platelets, with some degranulation. Serial flow cytometry in one experiment showed decreasing contamination of CD41 negative particles as CD41 positive small particles increased (FIG. 1C). The day 7 results for the platelet differentiation medium and the control medium were identical, as expected. At 14 days, a large population of small (low forward scatter) CD41 positive particles were recorded in the sample from the differentiation bioreactor; these were presumably platelets. There was also a significant population of small particles that were not CD41 positive, probably cellular debris. This debris almost disappeared at the day 21 and day 28 time points, while the small-sized CD41 positive population persisted.

The platelets collected aggregated in response to thrombin, as did neonatal control platelets. We analyzed the platelets for CD62 and CD 63 expression before and after thrombin exposure, and compared this to neonatal platelets harvested from cord blood within 24 hours of delivery, using flow cytometry (FIG. 1F). The bioreactor-produced platelets showed considerable CD62 and CD63 activation above that seen with cord blood platelets. Thrombin activation increased the expression of both markers of platelet activation above the baseline expression.

### Effect of Protein Coating Scaffold

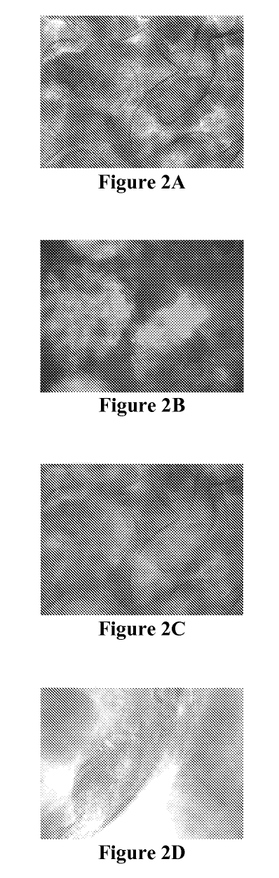

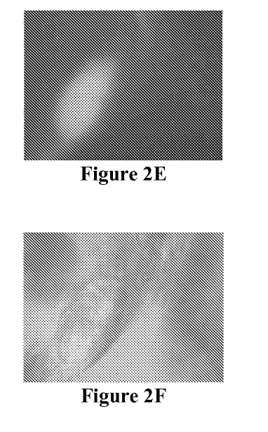

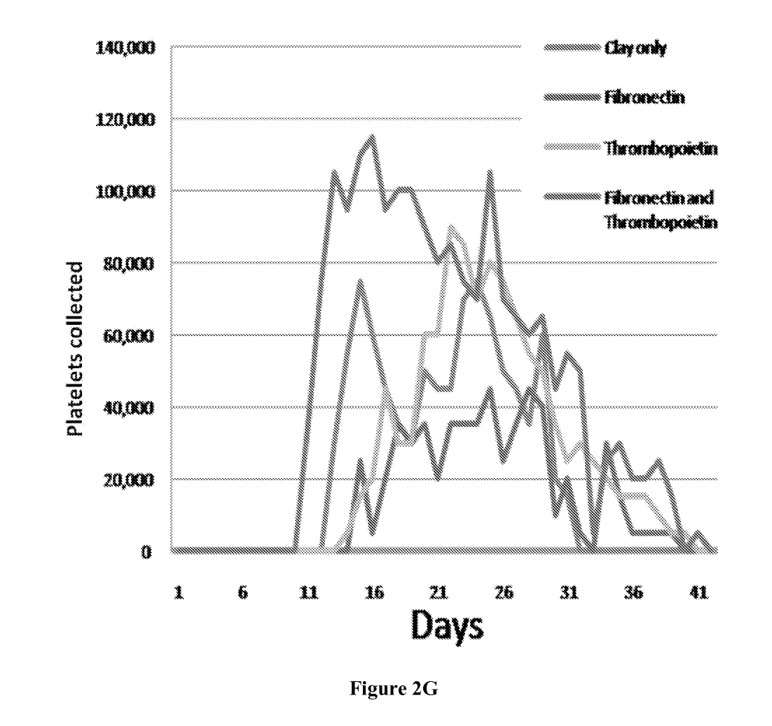

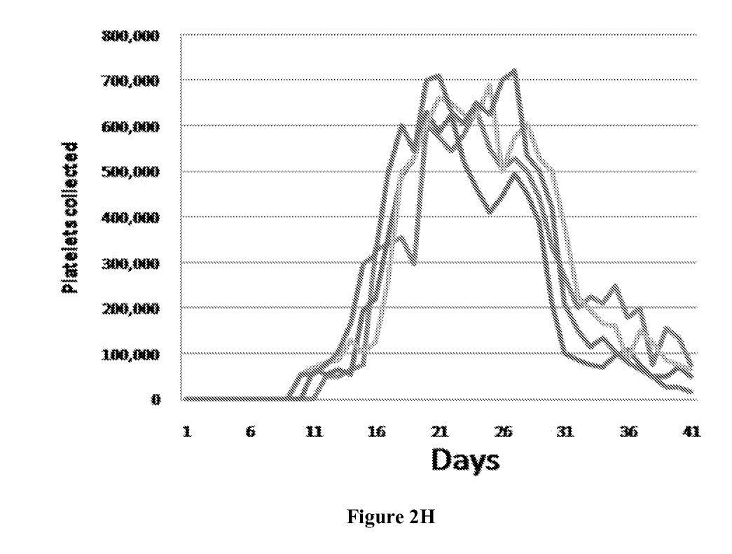

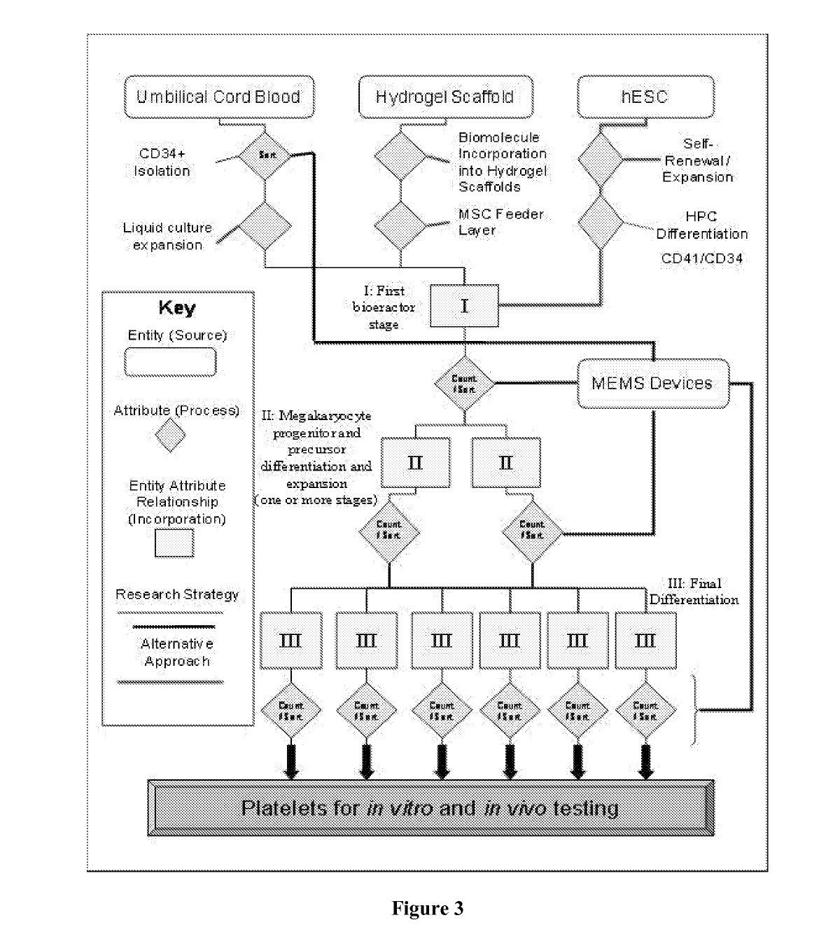

To see the potential effect of embedded proteins in 3D, we used hydrogel scaffolds coated with clay without embedded protein or decorated with fibronectin and/or thrombopoietin, in tissue culture wells (FIG. 2 and Table 1b). The results suggested fibronectin and TPO increased the amount and duration of platelet production, over plain clay coating alone (FIG. 2B). Increasing IL-6 and adding IL-11 increased platelet production, and the effect of protein coatings was largely lost (FIG. 2B). Total production normalized to starting expanded CD34 positively-selected cells approximately doubled (Table 1).

Flow/cross flow in the bioreactor system: We postulated that instituting continuous flow during incubation in the bioreactor would increase platelet production. We based this expectation on descriptions in the literature of up to a 20 fold increase in platelet production in a 2D flow system. [47, 45] We compared flow across the cell-scaffold construct achieved by blocking the lower medium input port and the upper output port with flow parallel to the cell-scaffold construct (all ports open). A 3 mL bolus was used for harvest in both the cross and continuous flow modes, and for the intermittent flow bolus alone method used previously. We found a marked increase in platelet production, up to a 3-fold increase from day 17 onward, with cross-flow but not parallel flow (FIG. 4).

We explored decreased oxygen level during culture to enhance platelet production. Our reasoning was that there is a lower level of oxygen in the marrow space near the surrounding bone where early hematopoietic cells are found. We compared 20% O₂ vs. 5% O₂. We found that if the entire platelet production scheme outlined above was performed at 5% O₂, there was a marked diminution in platelet production. On the other hand, if the initial 2-3 day expansion in liquid culture and the 7 day expansion in the 3D scaffold were performed at 5% and the differentiation stage was carried out at 20% O₂, we found an approximate doubling of the number of platelets produced in the 3D and 3D bioreactor systems.

### SUMMARY

In the disclosure presented here, we avoided the use of feeder cells. Some aspects of the 3D niche microenvironment can be supplied in a culture system in which a feeder cell layer is used. This feeder layer, often cell lines or MSCs, may supply points of contact for adherent cells, molecules that interact with receptors on the target cell's surface, or the opposite, and may also produce soluble chemokines or cytokines necessary for the target cells growth and survival [15-17]. Osiris Therapeutics, Inc., has reported successfully growing marrow CD34 positive cells on MSCs, with formation of megakaryocyte colonies in serum-free medium without added cytokines [18]. Because of the difficulty of obtaining and maintaining a feeder layer in a large scale system, we sought, successfully, to determine whether we could produce functional platelets using a feeder-free 3D culture system.

We demonstrated marked improvement in platelet production when 3D cell-scaffold constructs were created and grown in a 3D modular single-pass perfusion bioreactor system. Several types of bioreactors have been described for growth of hematopoietic cells. The most simple, suspension or 2D adherent-cell cultures, allow minimum cell-cell contact. Most flow systems such as packed bed reactors produce significant shear forces on the growing cells [32-34]. The Aastrom RepliCell, initially introduced to expand marrow-derived progenitors, uses a single pass 2D system [35]. Other 3-dimensional culture methods for human cells that utilize some form of bioreactor include a tantalum-coated porous biomaterial (Cytomatrix) to culture and expand hematopoietic progenitor cells from bone marrow for up to 6 weeks and umbilical cord blood CD34+ cells for up to 2 weeks [36, 37]. Banu et al, successfully cultured CD34+ cells from human bone marrow on a porous three-dimensional biomatrix (Cellfoam™) for up to 6 weeks [38]. Zhao and Ma reported the used PET matrix scaffolds in a bioreactor device to culture human mesenchymal stem cells (MSCs) for a period of 40 days [39]. Braccini et al, also reported expansion of MSCs in a 3D scaffold-based bioreactor system, in addition to co-culture of hematopoietic progenitor cells [40]. Our bioreactor system is purpose-built for hematopoietic culture; it allows continuous collection of non-adherent cells. At the same time, it allows independent control of medium and gas flow, as well as a variety of medium utilization methods. The system as described here uses a discontinuous (every 24 h) single-pass scheme, but it can be configured for continuous or pulsed flow with or without automatic recycling of the output medium. The environment engendered by the bioreactor module, even with the limited (once per 24 hours) flow, allows nutrient, waste, and gas exchange above and below the 3D scaffold; we believe this accounts for the greatly increased platelet production in the bioreactor system.

To test function, we performed aggregation studies using a method for small numbers of platelets [28]. There was also a measurable response to thrombin using antibodies to CD62 and CD63 by flow cytometry. The flow studies, while showing an increment in activation with thrombin, also showed that these platelets were activated in the absence of added thrombin. Several possible explanations, including activation on contact with portions of the bioreactor system flow path or activation by the cytokine-containing differentiation medium, are possible. We also noted somewhat deranged morphology compared to similarly-prepared neonatal platelets in both Wright's stained preparations and with TEM. We think that the activation sans added agonists and the abnormal morphology can be remedied by several future changes in the system, notably by using continuous medium flow into the bioreactor modules with resulting continuous collection of shed platelets, and by modifying the surfaces of the bioreactor system and scaffolds.

To test platelet formation in the presence or absence of adherent TPO or fibronectin. We used poly(Am) hydrogel as the substrate material for an inverted crystalloid scaffold, chosen for its biocompatibility, mechanical strength and transparency. Mechanical rigidity resulted in a firm ICC structure and transparency allowed facile optical analysis. We modified the hydrogel surface using by layer-by-layer deposition of positively charged 0.5 wt % PDDA solution and negatively charged 0.5 wt % clay platelet dispersion [43]. The LBL deposition of nano-structured clay platelet and PDDA increased nano-scale roughness and created surface charge and stiff film, which worked in concert, along with adhered proteins, to bind and stimulate cells. We found that with the combination of both TPO and fibronectin, utilizing the SRC kinase inhibitor medium, platelet production lasted longer at a higher level vs. only one or the other or neither. On the other hand, when we maximized platelet production using IL-6 and IL-11, little increase was seen in platelet numbers produced with either or both coating proteins. Our hypothesis is that the TPO has both a stimulating and a binding effect in our system, and that fibronectin also binds both progenitors and megakaryocytes during hematopoiesis in the system. The need for the bound proteins to increase platelet production is overcome by increasing fluid-phase IL-6 and adding IL-11.

The current results show continuous prolonged production of platelets using a 3D single-pass intermittent-flow perfusion bioreactor system, markedly more than with conventional 2D culture. While we expected to produce platelets with the system, its ability to produce them over a long period of time (several weeks) was unexpected. It appears that the 3D milieu engendered by the scaffolds we have used, especially when used with our modular bioreactor system, allows asynchronous production of platelet progenitors and precursors for prolonged periods. Even so, the number of platelets produced falls far short of the number needed for transfusion. Future improvements to increase yield include adoption of methods to increase the number of early progenitors from cord blood; longer periods of continuous perfusion, with or without medium recirculation; and alterations in scaffold surface makeup and design.

More than two million platelet transfusions are given in the US annually. The brief storage time of platelets, 5 to 7 days, often creates shortages in times of emergency and environmental crisis. These and other difficulties could be overcome with an in vitro platelet production system. The work presented here may provide a foundation for future development of such a clinical production system, in addition to creating an in vitro model of megakaryocytopoiesis and thrombopoiesis that can be used to study these processes and the effect of drugs and disease.

TABLE 1
a: Experimental summary. Platelet yields from start of CD34 selection (cell and platelet counts in millions).

| | | | | Platelets | Platelets | | |
|---|---|---|---|---|---|---|---|
| | | | Starting | from 2D | from 3D | | |
| | | | cell | ×10⁶ | ×10⁶ | Number of | Platelets from 3D |
| | After | After | number | (number | (number | bioreactor | Bioreactor |
| | CD34+ | liquid | per | per | per | modules/ | ×10⁶ |
| | Column | expansion | condition | starting | starting | Number of | (number per starting |
| Experiment | ×10⁶ | ×10⁶ | ×10⁶ | cell) | cell) | disks* | cell***) |
| 1 Src differentiation | 1.8 | 14 | 2.2 | 2.3 (1.0) | 10.5 (4.8) | 2/6 | 31.3 (14.2) |
| medium | | | | | | | |
| 2 Src differentiation | 1.5 | 18 | 2.8 | 1.7 (0.61) | 6.9 (2.5) | 2/6 | 30.7 (10.9) |
| medium | | | | | | | |
| 3 Src differentiation | 2.1 | 22 | 3.5 | 2.8 (0.80) | 9.3 (2.6) | 2/6 | 36.2 (10.3) |
| medium | | | | | | | |
| 1 | | | | | | | |
| IL-6, -11 medium | 1.3 | 12 | 6 | ND | ND | 4/12 | 117.7 (19.6) |
| Coated hydrogel | 1.5 | 11 | 2 | ND | 4.4** (2.2**) | ND | ND |
| scaffolds-- | | | | | | | |
| Src differentiation | | | | | | | |
| medium | | | | | | | |
| Coated hydrogel | 2.1 | 14 | 8 | ND | 38.1** (4.7**) | ND | ND |
| scaffolds | | | | | | | |
| 3 stage | | | | | | | |

b: Coated hydrogel scaffold experiments. Platelet yields from start of CD34 selection (cell and platelet counts in
millions).

| | After | After | | | | | | |
|---|---|---|---|---|---|---|---|---|
| | CD34+ | liquid | Starting | Clay | | TPO | FN + TPO | Total Platelets from |
| Experiment | column | expansion | number | Coated | FN Coated | Coated | Coated | all 4 conditions |
| Coated | 1.5 | 11 | 2 | 0.9 | 0.6 | 1.0 | 1.9 | 4.4 |
| hydrogel | | | (0.5 per | | | | | (2.2 per starting cell) |
| scaffolds-- | | | scaffold, 1 | | | | | |
| Src | | | scaffold | | | | | |
| differentiation | | | disk per | | | | | |
| medium | | | condition) | | | | | |
| Coated | 2.1 | 14 | 8 | 9.3 | 8.1 | 10.2 | 10.5 | 38.1 |
| hydrogel | | | (0.5 per | (2.3 per | (2.0 per | (2.5 per | (2.6 per | (9.5 per 4 scaffolds) |
| scaffolds-- | | | scaffold, 4 | scaffold) | scaffold) | scaffold) | scaffold) | (4.7 per starting cell) |
| 3 stage | | | scaffold | | | | | |
| | | | disks per | | | | | |
| | | | condition) | | | | | |

*Number of disks applies to both the 3D and the 3D Bioreactor conditions. Six wells in 12 well plates were used for eache 2D condition.
**Total for 4 conditions-see Table 1b
***For experiments 1 to 3, p < .05 for 3D Bioreactor vs. 2D or 3D using paired t-test. Paired T-test for 2D vs 3D-normalized to platelets per starting cell p = .062 (SPSS, Inc., Chicago, Illinois).
Hydrogel scaffolds were 15 mm diameter disks approximately 2 mm in thickness.

### REFERENCES

[1] Matsunaga T TI, Kobune M, Kawano Y, Tanaka M, Kuribayashi K, Iyama S, Sato T, Sato Y, Takimoto R, Takayama T, Kato J, Ninomiya T, Hamada H, Niitsu Y. Ex Vivo Large-Scale Generation of Human Platelets from Cord Blood CD34+ Cells. Stem Cells. 2006 24:2877-2887.
[2] Liu Y L T, Fan X, Ma X, Cui Z. Ex vivo expansion of hematopoietic stem cells derived from umbilical cord blood in rotating wall vessel. J Biotechnol. 2006; 124:592-601.
[3] De Bruyn C, Delforge A, Martiat P, Bron D. Ex Vivo Expansion of Megakaryocyte Progenitor Cells Cord Blood Versus Mobilized Peripheral Blood. Stem Cells and Development. 2005; 14:415-424.
[4] Yao C L CI, Hsieh T B, Hwang S M. A systematic strategy to optimize ex vivo expansion medium for human hematopoietic stem cells derived from umbilical cord blood mononuclear cells. Exp Hematol. 2004; 32:720-727.
[5] Shim M H HA, Blake N, Drachman J G, Reems J A. Gene expression profile of primary human CD34+ CD3810 cells differentiating along the megakaryocyte lineage. Exp Hematol. 2004; 32:638-648.
[6] Kawano Y K M, Yamaguchi M, Nakamura K, Ito Y, Sasaki K, Takahashi S, Nakamura T, Chiba H, Sato T, Matsunaga T, Azuma H, Ikebuchi K, Ikeda H, Kato J, Niitsu Y, Hamada H. Ex vivo expansion of human umbilical cord hematopoietic progenitor cells using a coculture system with human telomerase catalytic subunit (hTERT)-transfected human stromal cells. Blood. 2003; 101:532-540.
[7] Schipper L F B A, Reniers N, Melief C J, Willemze R, Fibbe W E. Differential maturation of megakaryocyte progenitor cells from cord blood and mobilized peripheral blood. Exp Hematol. 2003; 31:324-330.
[8] Sabri S J-P M, Bertoglio J, Farndale R W, Mas V M, Debili N, Vainchenker W. Differential regulation of actin stress fiber assembly and proplatelet formation by alpha2beta1 integrin and GPVI in human megakaryocytes. Blood. 2004; 104:3117-3125.
[9] Fujimoto T T, Kohata S, Suzuki H, Miyazaki H, Fujimura K. Production of functional platelets by differentiated embryonic stem (ES) cells in vitro. Blood. 2003; 102:4044-4051.
[10] Eto K, Murphy R, Kerrigan S W, et al. Megakaryocytes derived from embryonic stem cells implicate CalDAG-GEFI in integrin signaling. PNAS. 2002; 99 12819-12824.
[11] Gandhi M J, Drachman J G, Reems J A, Thorning D, Lannutti B J. A novel strategy for generating platelet-like fragments from megakaryocytic cell lines and human progenitor cells. Blood Cells Mol. Dis. 2005; 35:70-73.
[12] Mazur E M B D, Newton J L, Cohen J L, Charland C, Sohl P A, Narendran A. Isolation of large numbers of enriched human megakaryocytes from liquid cultures of normal peripheral blood progenitor cells. Blood. 1990; 76:1771-1782.
[13] Kaushansky K B V, Lin N, Jorgensen M J, McCarty J, Fox N, Zucker-Franklin D, Lofton-Day C. Thrombopoietin, the Mp 1 ligand, is essential for full megakaryocyte development. Proc Natl Acad Sci USA. 1995; 92:3234-3238.
[14] Adams G B, Scadden D T. The hematopoietic stem cell in its place. Nat Immunol. 2006; 7:333-337.
[15] Roecklein B A, Torok-Storb B. Functionally distinct human marrow stromal cell lines immortalized by transduction with the human papilloma virus E6/E7 genes. Blood. 1995; 85 997-1005.
[16] Kadereit S, Deeds L S, Haynesworth S E, et al. Expansion of LTC-ICs and maintenance of p21 and BCL-2 expression in cord blood CD34(+)/CD38(−) early progenitors cultured over human MSCs as a feeder layer. Stem Cells. 2002; 20:573-582.
[17] Cheng L, Hammond H, Ye Z, Zhan X, Dravid G. Human adult marrow cells support prolonged expansion of human embryonic stem cells in culture. Stem Cells. 2003; 21:131-142.
[18] Cheng L, Qasba P, Vanguri P, Thiede M A. Human mesenchymal stem cells support megakaryocyte and pro-platelet formation from CD34(+) hematopoietic progenitor cells. J Cell Physiol. 2000; 184:58-69.
[19] Li Y, Ma T, Kniss D A, Yang S T, Lasky L C. Human cord cell hematopoiesis in three-dimensional nonwoven fibrous matrices: in vitro simulation of the marrow microenvironment. J HematotherStem Cell Res. 2001; 10:355-368.
[20] Kotov N, Liu Y, Wang S, et al. Inverted Colloidal Crystals as 3D Cell Scaffolds Langmuir. 2004; 20 7887-7892.
[21] Lee J, Shanbhag S, Kotov N A. Inverted colloidal crystal scaffolds as three dimensional microenvironments to study cell interactions in co-cultures. JMaterRes. 2006; 16:3558-3564.
[22] Shanbhag S, Lee J W, Kotov N. Diffusion in three-dimensionally ordered scaffolds with inverted colloidal crystal geometry. Biomaterials. 2005; 26:5581-5585.
[23] Zhang Y, Wang S, Eghtedari M, Motamedi M, Kotov N A. Inverted-colloidal-crystal hydrogel matrices as three-dimensional cell scaffolds. Advanced Functional Materials. 2005; 15:725-731.
[24] Zweegman S VDBJ, Mus A M, Kessler F L, Janssen J J, Netelenbos T, Huijgens P C, Drager A M. Bone marrow stromal proteoglycans regulate megakaryocytic differentiation of human progenitor cells. Exp Cell Res. 2004; 299:383-392.
[25] Tablin F CM, Leven R M. Blood platelet formation in vitro. The role of the cytoskeleton in megakaryocyte fragmentation. J Cell Sci. 1990; 97 (Pt 1):59-70.
[26] Cabrita G J M, Ferreira B S, Lobato de Silva C, Goncalves R, Almeida-Porada G, Cabral JMS. Hematopoietic stem cells: from the bone to the bioreactor. Trends Biotechnol. 2003; 21 233-240.
[27] Cuddihy J M, Kotov N A. Poly(lactic-co-glycolic acid) bone scaffolds with inverted colloidal crystal geometry. Tissue Engineering Part A; 2008.
[28] Holme. Improved in vivo and in vitro viability of platelet concentrates stored for seven days in a platelet additive solution. Br J Haematol. 1987 66:233-238.
[29] McLean M R L A, Garancis J C, Hause L L. A method for the preparation of platelets for transmission electron microscopy. Stain Technol. 1982; 57:113-116.
[30] Cortin V, Garnier A, Pineault N, Lemieux R, Boyer L, Proulx C. Efficient in vitro megakaryocyte maturation using cytokine cocktails optimized by statistical experimental design. Exp Hematol. 2005; 33:1182-1191.
[31] Fujiki. Role of human interleukin-9 as a megakaryocyte potentiator in culture. Exp Hematol 2002; 30:1373-1380.
[32] Mantalaris A, Keng P, Bourne P, Chang A Y, Wu J H. Engineering a human bone marrow model: a case study on ex vivo erythropoiesis. Biotechnol Prog. 1998; 14:126-133.
[33] Meissner P, Schroder B, Herfurth C, Biselli M. Development of a fixed bed bioreactor for the expansion of human hematopoietic progenitor cells. Cytotechnology. 1999; 30:227-234.
[34] Jelinek N, Schmidt S, Hilbert U, Thoma S, Biselli M, Wandrey C. Novel Bioreactors for the ex vivo Cultivation of Hematopoietic Cells. Enginnering in Life Sciences. 2002; 2:15-18.
[35] Jaroscak J, Goltry K, Smith A, et al. Augmentation of umbilical cord blood (UCB) transplantation with ex vivo-expanded UCB cells: results of a phase 1 trial using the AastromReplicell System. Blood. 2003; 101:5061-5067.
[36] Bagley J, Rosenzweig M, Marks D F, Pykett M J. Extended culture of multipotent hematopoietic progenitors without cytokine augmentation in a novel three-dimensional device. Experimental Hematology. 1999; 27:496-504.
[37] Ehring B, Biber K, Upton T M, Plosky D, Pykett M, Rosenzweig M. Expansion of HPCs from cord blood in a novel 3D matrix. Cytotherapy. 2003; 5:490-499.
[38] Banu N, Rosenzweig M, Kim H, Bagley J, Pykett M. Cytokine-augmented culture of haematopoietic progenitor cells in a novel three-dimensional cell growth matrix. Cytokine 2001; 13:349-358.
[39] Zhao F, Ma T. Perfusion bioreactor system for human mesenchymal stem cell tissue engineering: dynamic cell seeding and construct development. Biotechnol Bioeng. 2005; 91:482-493.
[40] Braccini A, Wendt D, Jaquiery C, et al. Three-dimensional perfusion culture of human bone marrow cells and generation of osteoinductive grafts. Stem Cells. 2005; 23:1066-1072.
[41] Israels S J R M, Michelson A D. Neonatal platelet function. Semin Thromb Hemost. 2003; 29:363-372.
[42] Israels. Impaired signal transduction in neonatal platelets. Pediatr Res. 1999 45:687-691.
[43] Tang Z, Kotov N A, Magonov S, Ozturk B. Nanostructured artificial nacre. Nature Materials. 2003; 2:413-418.
[44] Molla A, Mossuz P, Berthier R. Extracellular matrix receptors and the differentiation of human megakaryocytes in vitro. Leuk Lymphoma. 1999; 33:15-23.
[45] Dunois-Larde, C., C. Capron, et al. (2009). “Exposure of human megakaryocytes to high shear rates accelerates platelet production.” Blood 114(9): 1875-83.
[46] Huang, C. H., P. M. Chen, et al. (2009). “Purified Recombinant TAT-HOXB4 expands CD34+ Umbilical Cord Blood and Peripheral Blood Progenitor Cells ex vivo.” Tissue Eng Part C Methods.
[47] Junt, T., H. Schulze, et al. (2007). “Dynamic visualization of thrombopoiesis within bone marrow.” Science 317(5845): 1767-70.
[48] Lu, S. J., Q. Feng, et al. (2007). “Recombinant HoxB4 fusion proteins enhance hematopoietic differentiation of human embryonic stem cells.” Stem Cells Dev 16(4): 547-59.
[49] Ohta, H., S. Sekulovic, et al. (2007). “Near-maximal expansions of hematopoietic stem cells in culture using NUP98-HOX fusions.” Exp Hematol 35(5): 817-30.
[50] Zhang (2007). “Effects of HOXB4 overexpression on ex vivo expansion and immortalization of hematopoietic cells from different species.” Stem Cells 25(8): 2074-81.

1. A method for platelet production comprising:
providing a cellular material; and
culturing the cellular material,
wherein platelets are produced.

2. The method of claim 1 wherein the culturing is performed on a two-dimensional cell support structure, three-dimensional cell support structure, or in suspension culture.

3. The method of claim 1 further comprising a step of isolating a subset of cells from the cellular material, wherein the isolated subset of cells are cultured, and wherein platelets are produced.

4. The method of claim 2 wherein the three-dimensional cell support structure is polymer-based, ceramic/thin film based, human derived, animal derived, or fabric-based.

5. The method of claim 4 wherein the polymer-based three-dimensional cell support structure is microporous or macroporous.

6. The method of claim 5 wherein the microporous or macroporous polymer-based three-dimensional cell support structure is colloidal crystal or polyacrylamide gel.

7. The method of claim 4 wherein the ceramic/thin film based three-dimensional cell support structure is selected from medical ceramic, plastic, and bioglass.

8. The method of claim 7 wherein the plastic three-dimensional cell support structure is selected from monostructured thermomolded monofilm, polymer thin films, and tissue culture plastics.

9. The method of claim 4 wherein the fabric-based three-dimensional cell support structure is woven or non-woven.

10. The method of claim 9 wherein the woven fabric-based three-dimensional cell support structure is BARD.

11. The method of claim 9 wherein the non-woven fabric-based three-dimensional cell support structure is PET.

12. The method of claim 1 wherein the cellular material is progenitor cellular material.

13. The method of claim 12 wherein the progenitor cellular material is pluripotent stem cells.

14. The method of claim 13 wherein the pluripotent stem cells are selected from the group consisting of derived stem cells, harvested stem cells, and embryonic stem cells.

15. The method of claim 14 wherein the harvested stem cells are selected from the group consisting of induced adult stem cells, fetal liver stem cells, and umbilical cord blood stem cells.

16. The method of claim 14 wherein the derived stem cells, harvested stem cells, and embryonic stem cells are selected from CD34+ or CD133+.

17. The method of claim 16 wherein the CD34+ and CD133+ are selected from the group consisting of hematopoietic stem cells (HSCs), multipotent progenitor cells (MPPs), and lineage-restricted progenitor cells (LRPs).

18. The method of claim 17 wherein the HSCs are CD150⁻CD48⁺CD244⁺.

19. The method of claim 17 wherein the LRPs are CD150⁻CD48⁺CD244⁺.

20. The method of claim 17 wherein the HSCs are selected from the group consisting of mouse HSC, human HSC, LT-HSC, and ST-HSC.

21. The method of claim 20 wherein the mouse HSC is CD34^{lo/-}, SCA-1⁺, Thy1.1^{+/lo}, CD38⁺, C-kit⁺, lin⁻.

22. The method of claim 20 wherein the human HSC is CD34⁺, CD59⁺, Thy1/CD90⁺, CD38^{lo/-}, C-kit/CD117⁺, lin⁻.

23. The method of claim 20 wherein the LT-HSC is CD34⁻, SCA-1⁺, Thy1.1^{-/lo}, C-kit⁺, lin⁻, CD135⁻, Slamf1/CD150⁺.

24. The method of claim 20 wherein the ST-HSC is SCA-1⁺, Thy1.1^{+/lo}, C-kit⁺, lin⁻, CD135⁻, Slamf1/CD150⁺, Mac-1 (CD11b)^{lo}.

25. The method of claim 17 wherein the MPPs are selected from early MPPs and late MPPs.

26. The method of claim 25 wherein the early MPPs are CD34⁺, SCA-1⁺, Thy1.1⁻, C-kit⁺, lin⁻, CD135⁺, Slamf1/CD150⁻, Mac-1 (CD11b)^{lo}, CD4^{lo}.

27. The method of claim 25 wherein the late MPPs are CD34⁺, SCA-1⁺, Thy1.1⁻, C-kit⁺, lin⁻, CD135^{high}, Slamf1/CD150⁻, Mac-1 (CD11b)^{lo}, CD4^{lo}.

28. The method of claim 1 wherein culturing the cellular material is performed under ex vivo culturing conditions.

29. The method of claim 28 wherein the ex vivo culturing conditions utilize a humidified atmosphere, maintained thermal conditions, and a cell culture media.

30. The method of claim 29 wherein the thermal conditions are selected from the group consisting of normothermic (37° C.), hyperthermic (>37° C.), and hypothermic (<37° C.).

31. The method of claim 29 wherein the atmospheric conditions are selected from the group consisting of: ambient humidified air, 5% CO₂; 5% O₂ humidified, 5% CO₂; and discontinuous 5% O₂ ambient humidified air, 5% CO₂.

32. The method of claim 29 wherein the cell culture media is selected to expand and differentiate the progenitor cellular material, and maintain and produce platelets.

33. The method of claim 32 wherein the cell culture media is Iscove's modified Dulbecco's medium (IMDM) based culture media comprising cytokines and chemical messengers to expand and differentiate the progenitor cellular material, and maintain and produce platelets.

34. The method of claim 29 wherein the cell culture media is selected from the group consisting of expansion media, maintenance media, differentiation media, and platelet production media.

35. The method of claim 34 wherein the expansion media comprises at least one of Iscove's modified Dulbecco's medium (IMDM), FCS, SCF, FL, TPO, IL-6, TAT-HOXB4, and NUP98-HOX.

36. The method of claim 35 wherein the expansion media comprises at least one of 10% fetal calf serum, 50 ng/mL SCF, 50 ng/mL FL, 10 ng/mL TPO, and 10 ng/mL IL-6.

37. The method of claim 34 wherein the expansion media is serum free.

38. The method of claim 37 wherein the serum free expansion media comprises at least one of Iscove's modified Dulbecco's medium (IMDM), SCF, FL, TPO, IL-6, TAT-HOXB4, and NUP98-HOX.

39. The method of claim 38 wherein the IMDM comprises at least one of 10% fetal calf serum, 50 ng/mL SCF, 50 ng/mL FL, 10 ng/mL TPO, and 10 ng/mL IL-6.

40. The method of claim 34 wherein the maintenance media comprises at least one of IMDM, FCS, HS, HC, TPO, and FL.

41. The method of claim 40 wherein the maintenance media comprises at least one of 10% FCS, 10% horse serum (HS), 0.25 uM hydrocortisone (HC), 10 ng/mL TPO, and 25 ng/mL FL.

42. The method of claim 34 wherein the maintenance media is serum free.

43. The method of claim 42 wherein the serum free maintenance media comprises at least one of IMDM, FCS, HS, HC, TPO, and FL.

44. The method of claim 43 wherein the serum free maintenance media comprises at least one of 10% FCS, 10% horse serum (HS), 0.25 uM hydrocortisone (HC), 10 ng/mL TPO, and 25 ng/mL FL.

45. The method of claim 34 wherein the differentiation media comprises at least one of IMDM, FBS, SRC, TPO, SCF, IL-6, and IL-9.

46. The method of claim 45 wherein the differentiation media comprises at least one of 10% FBS, 30 ng/mL TPO, 1 ng/mL SCF, 7.5 ng/mL IL-6, 13.5 ng/mL IL-9, and 2.5 uM SU6656.

47. The method of claim 34 wherein the differentiation media is serum free.

48. The method of claim 47 wherein the serum free differentiation medium comprises at least one of IMDM, FBS, SRC, TPO, SCF, IL-6, IL-9, and SU6656.

49. The method of claim 48 wherein the serum free differentiation medium comprises at least one of 10% FBS, 30 ng/mL TPO, 1 ng/mL SCF, 7.5 ng/mL IL-6, 13.5 ng/mL IL-9, and 2.5 uM SU6656.

50. The method of claim 34 wherein the platelet production media comprises at least one of IMDM, FBS, BSA, TPO, IL-3, IL-6, and IL-11.

51. The method of claim 50 wherein the platelet production media comprises at least one of 10% FBS, 1% bovine serum albumin (BSA), 25 ng/mL TPO, 25 ng/mL IL-3, 50 ng/mL IL-6, and 10 ng/mL IL-11.

52. The method of claim 34 wherein the platelet production media is serum free.

53. The method of claim 52 wherein the serum free platelet production media comprises at least one of IMDM, FBS, BSA, TPO, IL-3, IL-6, and IL-11.

54. The method of claim 53 wherein the serum free platelet production media comprises at least one of 10% FBS, 1% bovine serum albumin (BSA), 25 ng/mL TPO, 25 ng/mL IL-3, 50 ng/mL IL-6, and 10 ng/mL IL-11.

55. The method of claim 34 wherein the expansion medium is used in conjunction with a 5% O₂ atmosphere.

56. The method of claim 34 wherein the differentiation medium is used in conjunction with a 20% O₂ atmosphere.

57. The method of claim 1 wherein the culturing is performed in a bioreactor.

58. The method of claim 57 wherein the bioreactor is selected from the group consisting of stirred tank suspension, fixed/fluidized bed, airlift, perfusion chamber, hollow fiber, packed bed, rotating wall vessel, gas permeable storage bag, stirred vessel, BioFlo, Celligen, and WaveBag.

59. The method of claim 57 wherein the bioreactor is a perfusion bioreactor.

60. A method for platelet production comprising:
providing a cellular material;
isolating a subset of cells from the starting cellular material;
culturing the subset of cells;
seeding the subset of cells into a three-dimensional scaffold;
culturing the subset of cells in a three-dimensional scaffold;
seeding the cultured subset of cells into a bioreactor;
culturing the subset of cells in a bioreactor; and
harvesting the cells from the bioreactor,
wherein platelets are produced.

61. The method of claim 60 wherein any of the steps of isolating a subset of cells from the starting cellular material, culturing the subset of cells, seeding the subset of cells into a three-dimensional scaffold, culturing the subset of cells in a three-dimensional scaffold, seeding the cultured subset of cells into a bioreactor, culturing the subset of cells in a bioreactor, and harvesting the cells from the bioreactor may be repeated as necessary.

62. A platelet produced by a process comprising the steps of:
providing a cellular material; and
culturing the cellular material.

63. A platelet produced by a process comprising the steps of:
providing a cellular material;
isolating a subset of cells from the starting cellular material;
culturing the subset of cells;
seeding the subset of cells into a three-dimensional scaffold;
culturing the subset of cells in a three-dimensional scaffold;
seeding the cultured subset of cells into a bioreactor;
culturing the subset of cells in a bioreactor; and
harvesting the cells from the bioreactor.

64. The process of claim 63 wherein any of the steps of isolating a subset of cells from the starting cellular material, culturing the subset of cells, seeding the subset of cells into a three-dimensional scaffold, culturing the subset of cells in a three-dimensional scaffold, seeding the cultured subset of cells into a bioreactor, culturing the subset of cells in a bioreactor, and harvesting the cells from the bioreactor may be repeated as necessary.
